(19) United States
(12) Patent Application Publication
(10) Pub. No.: US 2024/0350734 A1
(43) Pub. Date: Oct. 24, 2024

(54) A method of assembling a medicament delivery device

(71) Applicant: SHL Medical AG, Zug (CH)

(72) Inventors: Shinshiuan Chiou, Zug (CH); Sebastian Karlsson, Zug (CH); Ming-Ting Yin, Taoyuan City (TW)

(21) Appl. No.: 18/688,187

(22) PCT Filed: Aug. 23, 2022
(86) PCT No.: PCT/EP2022/073528

§ 371 Date: Feb. 29, 2024

Related U.S. Application Data
(60) Provisional application No. 63/239,445, filed on Sep. 1, 2021.

(30) Foreign Application Priority Data
Oct. 1, 2021 (EP) 21200415.4

(51) Int. Cl. A61M 5/20; A61M 5/315; A61M 5/32; A61M 5/34

(52) U.S. Cl. CPC A61M 5/2033; A61M 5/31511; A61M 5/3158; A61M 5/3202; A61M 5/34; A61M 2207/10

(57) ABSTRACT

The present disclosure provides a method of assembling a medicament delivery device. The method includes providing a housing of the medicament delivery device, wherein the housing extends along a longitudinal axis between a proximal end and a distal end. The method also includes providing a power unit of the medicament delivery device, wherein the power unit comprises a plunger rod and a holder engaged with the plunger rod. The method also includes assembling the power unit into the housing. The method also includes providing a cap assembly extending along the longitudinal axis between a proximal end and a distal end. The method also includes connecting the cap assembly to either the plunger rod or the holder. The method also 10 includes moving the cap assembly relative to the housing in the distal direction of the housing until the cap assembly attaches to the proximal end of the housing, wherein during the step of moving the cap assembly relative to the housing, the cap assembly moves the plunger rod relative to the holder.

### TECHNICAL FIELD

The present disclosure generally relates to a method of assembling a medicament delivery device, and particularly to a method of assembling a medicament delivery device that comprises the step of attaching a cap assembly to a housing.

### BACKGROUND

Medicament delivery devices such as auto-injectors, inhalers and on-body devices are generally known for the self-administration of a medicament by patients without formal medical training. Therefore, those medicament delivery devices usually comprise multiple automatic functions and are usually delivered to end-users with prefilled medicament containers. A medicament container usually comprises a glass or plastic barrel with a stopper sealing on its rear end and a delivery member arranged on its front end or a pierceable membrane arranged on its front end. Due to the medicament container filling process and/or the characteristic of the contained medicament, some prefilled medicament containers also contain an amount of air. A priming shot is therefore needed to be carried out before a medicament delivery operation. Also, some medicament containers contain substances that need to be mixed before a medicament delivery operation.

It has been appreciated that improved solutions for providing a priming shot before a medicament delivery operation would be useful. It has also been appreciated that a new method of assembling that medicament delivery device would be beneficial.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a method of assembling a medicament delivery device, the method comprising: providing a housing of the medicament delivery device; wherein the housing extends along a longitudinal axis between a proximal end and a distal end; wherein the distal end of the housing is pointing away from a medicament delivery site during use of the medicament delivery device; and wherein the proximal end of the housing is pointing towards the medicament delivery site during use of the medicament delivery device; providing a power unit of the medicament delivery device; wherein the power unit comprises a plunger rod and a holder engaged with the plunger rod; assembling the power unit into the housing; providing a cap assembly extending along the longitudinal axis between a proximal end and a distal end; connecting the cap assembly to either the plunger rod or the holder; and moving the cap assembly relative to the housing in the distal direction of the housing until the cap assembly attaches to the proximal end of the housing, wherein during the step of moving the cap assembly relative to the housing, the cap assembly moves the plunger rod relative to the housing when the cap assembly is connected to the plunger rod; or the holder relative to the housing when the cap assembly is connected to the holder.

Another aspect of the invention provides a method of assembling a medicament delivery device, the method comprising: providing a housing of the medicament delivery device; wherein the housing extends along a longitudinal axis between a proximal end and a distal end; providing a power unit of the medicament delivery device; wherein the power unit comprises a plunger rod and a holder engaged with the plunger rod; assembling the power unit into the housing; providing a cap assembly extending along the longitudinal axis between a proximal end and a distal end; connecting the cap assembly to either the plunger rod or the holder; and moving the cap assembly relative to the housing in the distal direction of the housing until the cap assembly attaches to the proximal end of the housing, wherein during the step of moving the cap assembly relative to the housing, the cap assembly moves the plunger rod relative to the holder.

Preferably, according to another embodiment, before connecting the cap assembly to either the plunger rod or the holder, the method comprises the further steps of:

providing a medicament container; and inserting the medicament container into the housing from the proximal end of the housing.

Preferably, according to another embodiment, before assembling the power unit into the housing, the method comprises the further steps of providing a delivery member guard; and assembling both the delivery member guard and the power unit into the housing.

Preferably, according to another embodiment, the method comprises the further steps of engaging the delivery member guard to the holder either before or after assembling both the delivery member guard and the power unit into the housing.

Preferably, according to another embodiment, the step of moving the plunger rod relative to the housing comprises moving the plunger rod relative to the holder while the holder is immovable relative to the housing.

Alternatively, according to another embodiment, the step of moving the plunger rod relative to the holder comprises moving the plunger rod relative to the holder while the holder is immovable relative to the housing.

Preferably, according to another embodiment, during the step of moving the holder relative to the housing, the plunger rod is immovable relative to the housing.

Alternatively, according to another embodiment, the step of moving the plunger rod relative to the holder comprises moving the holder relative to the plunger rod while the plunger rod is immovable relative to the housing.

Preferably, according to another embodiment, the step of connecting the cap assembly to the plunger rod comprises the steps of contacting a proximal end of the medicament container with the cap assembly from the distal end of the cap assembly.

Preferably, according to another embodiment, the step of moving the plunger rod relative to the holder comprises the steps of using the cap assembly to move the medicament container in the distal direction relative to the housing; contacting a proximal end of the plunger rod with a distal end of the medicament container; using the distal end of the medicament container to move the plunger rod in the distal direction relative to the housing; disengaging the plunger rod from the holder by moving the plunger in the distal direction relative to the housing; and attaching the cap assembly to the proximal end of the housing.

Preferably, according to another embodiment, the step of connecting the cap assembly to the holder comprises the step of moving the cap assembly in the proximal direction relative to the delivery member guard, such that the cap assembly contacts a proximal end of the delivery member guard.

Preferably, according to another embodiment, the step of moving the plunger rod relative to the holder comprises the steps of: using the cap assembly to move the delivery member guard in the distal direction relative to the housing; using the distal movement of the delivery member guard to move the holder relative to the plunger rod; and attaching the cap assembly to the proximal end of the housing.

Preferably, according to another embodiment, the step of moving the holder relative to the plunger rod comprises a step of using the distal movement of the delivery member guard to rotate the holder relative to the plunger rod.

Preferably, according to another embodiment, the step of moving the holder relative to the plunger rod comprises a step of using the distal movement of the delivery member guard to linearly move the holder relative to the plunger rod in a direction transverse to the longitudinal axis.

Preferably, according to another embodiment, when the cap assembly is attached to the proximal end of the housing, the cap assembly moves axially towards the distal end of the housing relative to the housing until the cap assembly snap-fits to the housing.

Preferably, according to another embodiment, after the cap assembly is attached to the proximal end of the housing, the medicament delivery device is functionally completed.

Preferably, according to another embodiment, the medicament delivery device is an injection device, an inhalation device, or a medical sprayer.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1A schematically shows a perspective view of a medicament delivery device.

FIG. 1B schematically shows a cross-section view of a rear sub-assembly of the medicament delivery device of FIG. 1A.

FIG. 2A schematically shows a perspective view of components of the rear sub-assembly of FIG. 1B.

FIG. 2B schematically shows a perspective view of components of a cap assembly of the medicament delivery device of FIG. 1A.

FIG. 2C schematically shows a perspective view the cap assembly of FIG. 2B without a cover.

FIG. 2D schematically shows a cross-section view of the cap assembly of FIG. 2B

FIG. 3A schematically shows a cross-section view of a plunger rod of the rear sub-assembly of FIG. 1B, a plunger rod spring of the rear sub-assembly of FIG. 1B and a holder of the rear sub-assembly of FIG. 1B.

FIG. 3B schematically shows a cross-section view of the plunger rod of FIG. 3A and the holder of FIG. 3A.

FIG. 3C schematically shows a perspective view of the holder of FIG. 3A and a delivery member guard of the rear sub-assembly of FIG. 1B.

FIG. 4A schematically shows a side view of the rear sub-assembly of FIG. 1B and the cap assembly of FIG. 2B.

FIGS. 4B-C schematically show perspective views of the holder of FIG. 3A and the delivery member guard of the rear sub-assembly of FIG. 1B.

FIG. 4D schematically shows a cross-section view of the plunger rod of FIG. 3A and the holder of FIG. 3A.

FIG. 5A schematically shows a perspective view of the rear sub-assembly of FIG. 1B without the delivery member guard and a medicament container.

FIG. 5B schematically shows a cross-section view of a distal part of the rear sub-assembly of FIG. 5A.

FIG. 6A schematically shows a cross-section view of the cap assembly of FIG. 2B.

FIG. 6B schematically shows a perspective view of a medicament delivery device.

FIG. 6C schematically shows a cross-section view of a distal part of the rear sub-assembly of FIG. 6B.

### DETAILED DESCRIPTION

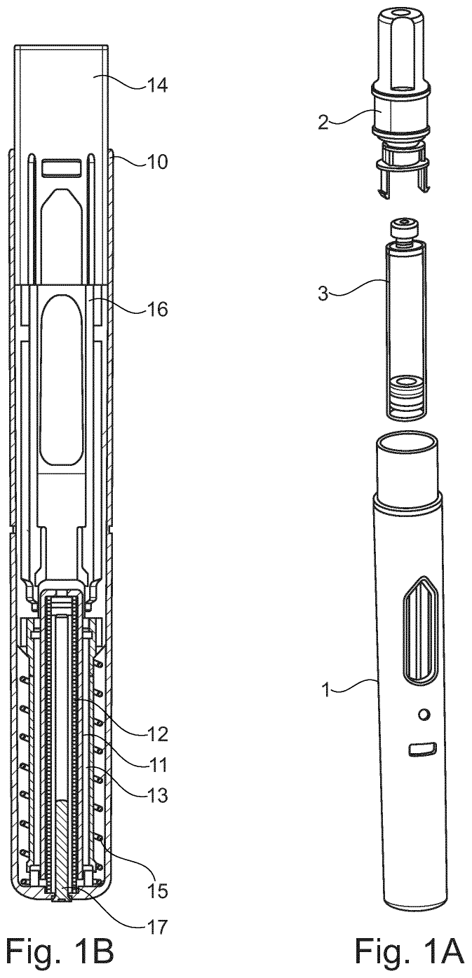

FIGS. 1A-6C illustrate a medicament delivery device which will be used to describe a method of assembly. The medicament delivery device comprises a rear sub-assembly 1, a cap assembly 2, and a medicament container 3. The rear sub-assembly 1 is the powerpack of the medicament delivery device. The powerpack is configured to expel a medicament contained within the medicament container 3 of the medicament delivery device when the medicament delivery device is in use. In one preferred example, the medicament delivery device is an auto-injector comprising an automatic function of priming and/or mixing substances from multiple chambers within a single medicament container. Alternatively, the medicament delivery device can be an inhalation device or a medical sprayer.

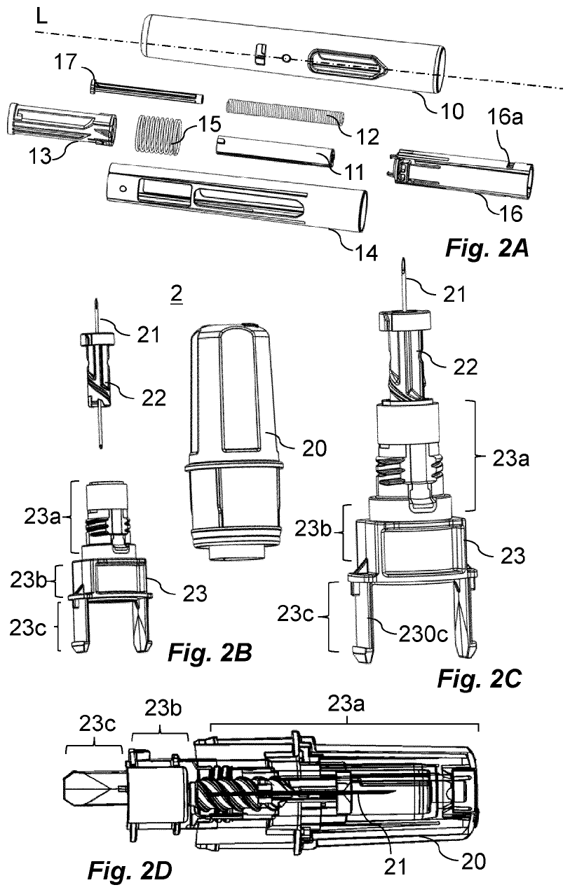

The rear sub-assembly 1, as shown in FIG. 1B and FIG. 2A, comprises a housing 10 extending along a longitudinal axis L between a proximal end and a distal end, a plunger rod 11, a stressed plunger rod spring 12 engaged with the plunger rod 11, a holder 13, and a delivery member guard 14 telescopically arranged relative to the proximal end of the housing 10. As above-mentioned definition, the distal end of the housing 10 is pointing away from a medicament delivery site during use of the medicament delivery device; and wherein the proximal end of the housing 10 is pointing towards the medicament delivery site during use of the medicament delivery device. The rear sub-assembly 1 further comprises a delivery member guard biasing member 15 arranged between a distally directed surface of the delivery member guard 14 and a proximally directed surface of the housing 10; optionally, a guide rod 17 is positioned within the plunger rod spring 12. The housing optionally comprises a medicament container holder 16 fixed to the housing 10. The medicament container holder 16 is configured to at least partially enclose the medicament container 3. The housing 10 further comprises a fastener configured to engage with the cap assembly 2 during the assembling process. The fastener can be arranged on a wall of the housing; or in an example where the housing 10 comprises the medicament container holder 16, the fastener 16a can be arranged on a wall of the medicament container holder 16.

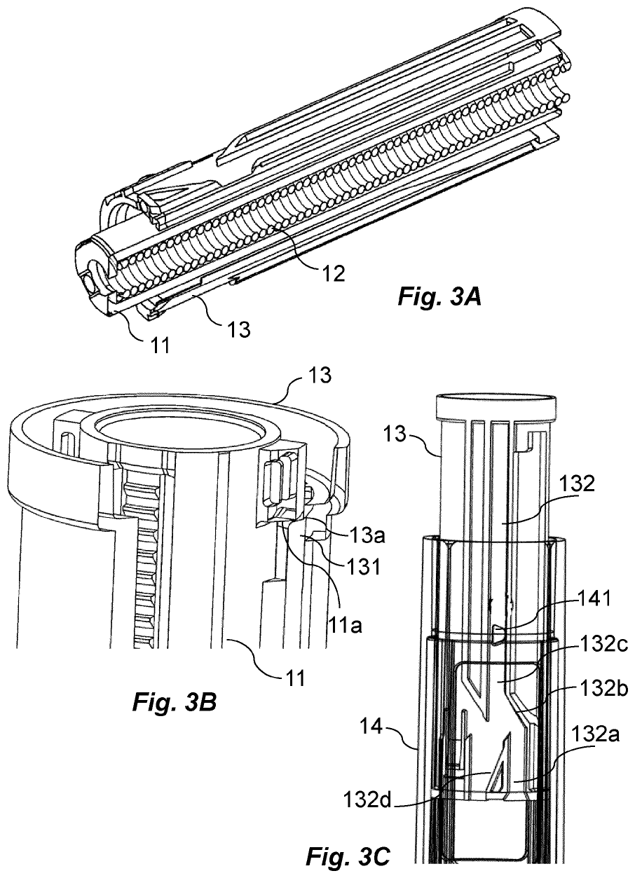

The plunger rod 11, the stressed plunger rod spring 12, and the holder 13 are all arranged within the housing 10. In an example where the rear sub-assembly comprises the delivery member guard biasing member 15 and the guide rod 17, the delivery member guard biasing member 15 and the guide rod 17 are both arranged within the housing 10. In an example where the housing 10 comprises a medicament container holder 16, the medicament container holder 16 is also arranged within the housing 10. The stressed plunger rod spring 12 is engaged with the plunger rod 11 at one end, and with the housing 10, e.g. directly or indirectly, at the other end, so that the stressed plunger rod spring 12 is configured to actuate the plunger rod 11 in the proximal direction of the housing 10 to expel a medicament contained within the medicament container 3 after the rear sub-assembly 1, the cap assembly 2, and the medicament container 3 are assembled together. When the plunger rod 11 is in a start position relative to the holder, the holder 13 is configured to releasably hold the plunger rod 11 against a biasing force from the plunger rod spring 12. As shown in FIG. 3B, the plunger rod 11 comprises a proximally directed holding surface 11a that is engaged with a distally directed counter holding surface 13a of the holder 13; and the distally directed counter holding surface 13a is generally perpendicular to the longitudinal axis L. In one example, the plunger rod spring 12 is arranged at least partially within the plunger rod 11, as shown in FIG. 3A. In another example, the plunger rod spring surrounds at least a portion of the plunger rod. In one example, the holder 13 is a rotator rotatable relative to the plunger rod 11 around the longitudinal axis L. The rotator comprises a generally tubular body, and a ledge (or alternatively a recess or cut-out) arranged on a surface of the tubular body for defining the distally directed counter holding surface 11a. In another example, the holder is a slider linearly movable relative to the plunger rod in a direction transverse to the housing. The holder comprises a generally tubular body, and a ledge (or alternatively a recess or cut-out) arranged on a surface of a wall for defining the distally directed counter holding surface. The holder 13 is configured to connect to a trigger of the medicament delivery device and is configured to be moved by the trigger to release the plunger rod from an original position so that the plunger rod moves in the proximal direction of the housing under the biasing force from the plunger rod spring to thus expel the contained medicament of the medicament delivery device. For example, when the medicament delivery device is configured to be activated by a movement of the delivery member guard 14 (the delivery member guard acts as the trigger), the holder is then connected to the delivery member guard; in another example, when the medicament delivery device is configured to be activated by a movement of a button such as a thumb button (the thumb button acts as the trigger), the holder is then connected to the button.

The cap assembly 2 comprises a cover 20 extending along the longitudinal axis L between a proximal end (closed end) and a distal end (open end). The cover 20 is configured to cover the medicament delivery member, e.g., a needle of the medicament delivery device when the medicament delivery device is an injector, before a medicament delivery operation, namely, before use of the medicament delivery device. As the medicament delivery member is arranged at the proximal end of the medicament delivery device, the cover 20 is configured to be at least partially attached to the medicament delivery device. In a preferred example, the cover is configured to cover a needle. In a preferred example, the cover 20 comprises a sleeve-shaped body extending between the proximal end and the distal end. Alternatively, the cover comprises a flat, button-shaped body, in this example, the medicament delivery member is mainly received within the housing of the medicament delivery device, thus, the cover is configured to cover the proximal end tip of the medicament delivery device. In one example, the cap assembly comprises a delivery member shield remover arranged within the cover. In an example where the medicament delivery device is an autoinjector with a medicament container comprising a rigid needle shield and/or flexible needle shield, the delivery member shield remover of the cap assembly is configured to fixedly engage with at least one of a rigid needle shield and a flexible needle shield of the medicament container. In this example, the medicament container is a syringe (a medicament container with an integral needle or nozzle on the medicament container). In another example, as shown in FIGS. 2B-D, the cap assembly 2 comprises the cover and a base 23, and optionally a delivery member 21 and a delivery member holder 22. The delivery member 21, the delivery member holder 22, and the base 23 are all arranged at least partially within the cover 20 and are all movable relative to the cover 20. The delivery member 21 is fixed to the delivery member holder 22. The delivery member 21 comprises a distal sharp end and a proximal delivery end. In this example, the medicament container 3 is a cartridge (a medicament container without an integral needle or nozzle on the medicament container). The distal shape end of the delivery member 21 is configured to penetrate a proximal seal of the medicament container 3 upon being activated so as to establish fluid communication between the medicament container and the proximal delivery end of the delivery member 21. The proximal delivery end of the delivery member 21 is configured to deliver the contained medicament to an end-user. For example, the proximal delivery end of the delivery member 21 can be a needle or a spray nozzle. The base 23 comprises a proximal portion 23a, a distal portion 23b and a counter fastener 23c. The delivery member holder 22 and the delivery member 22 are received within the proximal portion of the base 23. The counter fastener 23c is configured to engage with the fastener 16 of the housing 10 during the assembling process of the medicament delivery device. In a preferred example, the fastener 16a and the counter fastener 23c form a snap-fit engagement. In a preferred example, when the cap assembly 2 comprises the delivery member 21 and delivery member holder 22, the establishment of fluid communication between the medicament container and the proximal delivery end of the delivery member 21 is activated by the removal of the cover 20. On the other hand, in an example where the cap assembly 2 doesn't comprise the delivery member 21 and delivery member holder 22, then the end-user can establish fluid communication between the medicament container and a delivery member by attaching a delivery member, e.g. attaching a pen needle.

It should be noted that the description of this invention will not explain how to activate the fluid communication between the medicament container and the proximal delivery end of the delivery member by the removal of the cover in detail, because there are many different ways of activating the fluid communication between the medicament container and the proximal delivery end of the delivery member (see e.g. the disclosure of WO2009150078), and the how to activate the fluid communication between the medicament container and the proximal delivery end of the delivery member is not a part of the invention. Similarly, the description of this invention will not explain how to trigger a medicament delivery operation (completely releasing the plunger rod from the holder) in detail, because there are many different ways of triggering a medicament delivery operation (see e.g. WO2006057604), and the how to trigger a medicament delivery operation is not a part of the invention.

The method of the invention comprises the steps of providing the housing 10 of the medicament delivery device; and providing a power unit of the medicament delivery device. The power unit comprises the plunger rod 11 and the holder 13 engaged with the plunger rod 11. Assembling the power unit into the housing 10. Thus, the rear sub-assembly 1 has been assembled. The method of the invention comprises the steps of providing the cap assembly 2 extending along the longitudinal axis L between a proximal end and a distal end; connecting the cap assembly 2 to either the plunger rod 11 or the holder 13; and moving the cap assembly 2 relative to the housing 10 in the distal direction of the housing 10 until the cap assembly 2 attaches to the proximal end of the housing 10. During the step of moving the cap assembly 2 relative to the housing 10, the cap assembly 2 moves the plunger rod 11 relative to the holder 13.

In one example, the step of moving the plunger rod relative to the holder comprises a step of moving the holder 13 actively relative to the plunger rod 11. In this example, as shown in FIG. 3C, the holder 13 comprises a guiding structure 132 arranged on a wall of the holder 13. The delivery member guard 14 comprises a guide follower 141 configured to move along the guiding structure 132 of the holder 13. The guide follower 141 can be a protrusion extending from a surface of the delivery member guard 14 in a direction transverse to the longitudinal axis; preferably, extending from a distal end surface of the delivery member guard 14. In this example, the delivery member guard 14 is only movable relative to the housing in the axial direction. When the guide follower 141 moves along the guiding structure 132 of the holder 13, the axial movement of the guide follower 141 is configured to cause the holder 13 to rotate relative to the plunger rod 11 (if the holder 13 is the rotator); or to cause the holder 13 to move linearly relative to the plunger rod 11 in a direction transverse to the longitudinal axis L (if the holder is the slider). The guiding structure 132 can be a labyrinth formed by multiple ledges (or alternatively a recess or cut-out) on the wall of the holder 13. In an example where the holder 13 is the rotator, the labyrinth comprises two portions 132b, 132d helically extending on the tubular body of the rotator; therefore, when the guide follower 141 of the delivery member guard 14 moves along the portions 132b, 132d, the rotator will rotate relative to the plunger rod 11. On the other hand, in an example where the holder is the slider, the labyrinth comprises diagonal portions; as a result, when the guide follower of the delivery member guard moves along the diagonal portions, the slider will linearly move relative to the plunger rod 11 in a direction transverse to the longitudinal axis L. It should be noted that the diagonal portions or the helically extending portions 132b, 132d can also be formed at an edge of the slider or the rotator respectively.

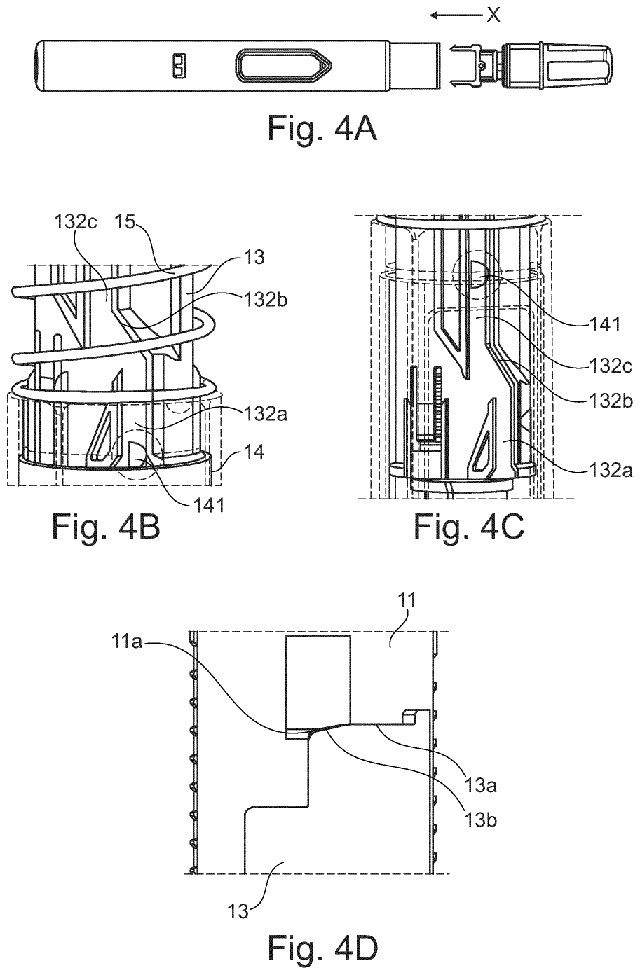

In one example, as shown in FIG. 4B, before attaching the cap assembly 2 to the proximal end of the housing 10, the guide follower 141 is positioned on a pre-assembled portion 132a of the guiding structure 132. In a preferred example, the delivery member guard biasing member 15 has already been assembled between the housing 10 and the delivery member guard 14. In this example, the delivery member guard 14 protrudes from the proximal end of the housing 10.

The next step is contacting the delivery member guard 14 with the cap assembly 2. In a preferred example, the distal end of the cover 20 of the cap assembly 2 is aligned with the proximal end of the housing 10, and the distal end of the cover 20 of the cap assembly 2 contacts a proximal end of the delivery member guard 14. Thereafter, the delivery member guard 14 is moved in the distal direction relative to the housing 10 by the cap assembly 2. The distal movement of the delivery member guard 14 causes the guide follower 141 to move along one of the diagonal portions or the helically extending portions 132b, 132d of the guiding structure 132, so that the holder 13 is rotated or moved linearly relative to the plunger rod 11, until the fastener 16a of the housing 10 engages with the counter fastener 23c of the cap assembly 2. The plunger rod 11 is now in the initial position relative to the holder 13. The delivery member guard 14 is then moved into a retracted position and thereby compresses the delivery member guard biasing member 15. The guide follower 141 is thereby positioned on an intermediate position 132c of the guide structure 132 of the holder 13. Therefore, when the end-user wants to use the medicament delivery device, the end-user will first remove the cover 20 from the medicament delivery device. Once the cover 20 has been removed, the delivery member guard 14 will move, under the biasing force of the delivery member guard biasing member 15, in the proximal direction relative to the housing 10. The guide follower 141 is thereby moved along the other one of the diagonal portions or the helically extending portions 132b, 132d of the guiding structure 132, so that the plunger rod 11 is released from the initial position and moves to the start position. The initial operation of the medicament delivery device is therefore performed. When the plunger rod 11 is in the start position, the plunger rod 11 can engage with the holder 13 or any other suitable component for holding the plunger rod against the force of the plunger rod spring before the medicament delivery operation. In a preferred example, the holder 13 comprises a second distally directed counter holding surface 13b, as shown in FIG. 4D. The second distally directed counter holding surface 13b extends from one end of the distally directed counter holding surface 13a in a direction circumferential to the longitudinal axis L, and is angled, e.g. between 10 and 80 degrees, relative to the distally directed counter holding surface 13a. In this example, the guide follower 141 is configured to block the rotation or the linear, transverse movement of the holder 13 (dependent on whether the holder 13 is a rotator or a slider) when the guide follower 141 is positioned in the intermediate position 132c of the guide structure 132. For example, the guide structure 132 may comprise one or more ledges abutting the guide follower 141 in a direction transverse to the longitudinal axis L. In this example, when the plunger rod 11 is in the original position, the proximally directed holding surface 11a is engaged with the distally directed counter holding surface 13a. During the assembling step of moving the holder 13 relative to the plunger rod 11, the plunger rod 11 moves to the initial position, and the proximally directed holding surface 11a is thus engaged with the second distally directed counter holding surface 13b. In this example, since the second distally directed counter holding surface 13b is a ramp surface, once the guide follower 141 moves out from the intermediate position 132c of the guide structure 132, the holder 13 will be moved by the plunger rod 11 under the force of the plunger rod spring 12. The delivery member guard biasing member 14 thus doesn't need to be too powerful; and the holder can, dependent on the selection of the angle of the second distally directed counter holding surface, only comprise one diagonal portion or the helically extending portion, because the holder now can be moved by the engagement between the second distally directed counter holding surface and the plunger rod. In this example, the medicament container 3 can be a cartridge or a syringe; the medicament container 3 can be assembled into the housing of the rear sub-assembly from the proximal end of housing 10, or from the distal end of the housing. In a preferred example, before attaching the cap assembly 2 to the proximal end of the housing 10, the method comprises the further steps of: providing a medicament container 3; and inserting the medicament container 3 into the housing from the proximal end of the housing 10. Therefore, the step of attaching the cap assembly 2 is the final step of assembling the medicament delivery device, meaning that the medicament delivery device is ready to be used by the end-user, from a functional perspective (the medicament delivery device might further need to be labelled and/or packed into a package from a regulation/sale aspect).

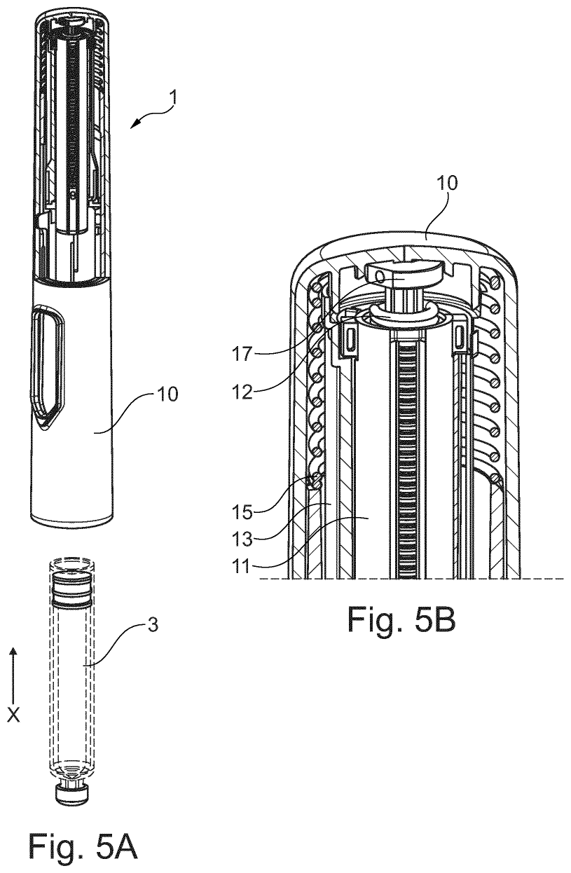

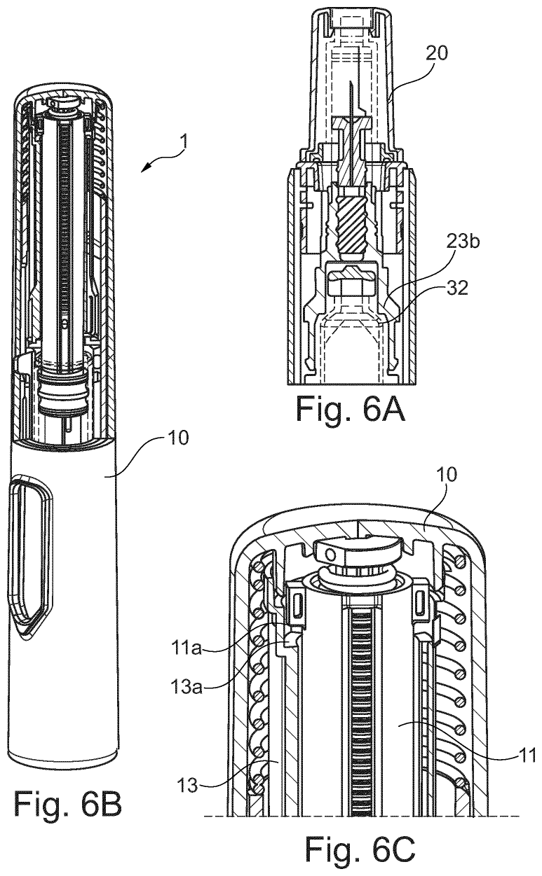

In another example, the step of moving the plunger rod 11 relative to the holder 13 comprises a step of moving the plunger rod 11 actively relative to the holder 13. This example will be suitable for assembling a medicament delivery device that comprises a cartridge; and with the assembling step that, before attaching the cap assembly 2 to the proximal end of the housing 10, the method comprises further steps of providing a medicament container 3; and inserting the medicament container 3 into the housing from the proximal end of the housing 10, as shown in FIG. 5A. In this example, when the rear sub-assembly 1 is provided, the plunger rod 11 is in the original position, meaning that the proximally directed holding surface 11a engages with a distally directed counter holding surface 13a of the holder 1, as shown in FIG. 5B. During assembly, the distal end of the cover 20 of the cap assembly 2 is aligned with the proximal end of the housing 10. The step of moving the plunger rod 11 relative to the holder 13 comprises the steps of contacting a proximal end of the medicament container 3 with the distal portion 23b of the base 23, as shown in FIG. 6A, in a preferred example, thereby contacting the proximal shoulder portion with the distal portion 23b of the base 23. Therefore, the step of attaching the cap assembly 2 to the proximal end of the housing can also push the plunger rod 11 in the distal direction relative to the holder 13, against the force of the plunger rod spring 12. The step further comprises moving the medicament container 3 in the distal direction relative to the housing 10 with the cap assembly 2; contacting a proximal end of the plunger rod 11 with a distal end of the medicament container 3, as shown in FIG. 6B; moving the plunger rod 11 in the distal direction relative to the housing 10 with the distal end of the medicament container 3; and moving the proximally directed surface 11a of the plunger rod 11 apart from the distally directed counter holding surface 13a of the holder 13 in the direction of the longitudinal axis L, as shown in FIG. 6C. The cartridge is a sealed medicament container, thus, before a fluid communication has been established between the medicament container and the delivery member, the contained medicament cannot be expelled from the medicament container. Therefore, when the cap assembly 2 has been attached to the proximal end of the housing 10, the distal portion 23b of the base 23 pushes on the medicament container to move the plunger rod 11 in the distal direction relative to the holder 13 to the initial position of the plunger rod 11; the plunger rod 11 is blocked from moving back to the original/start position by the medicament container thereafter. When the end-user establishes a fluid communication between the medicament container and the delivery member 21 by either removing the cover 20 or attaching a delivery member, e.g. a pen needle, since the medicament container is no longer sealed, it can therefore no longer block the plunger rod 11 against the force of the plunger rod spring 12, and the plunger rod 11 therefore moves in the proximal direction relative to the holder 13 until the proximally directed surface 11a of the plunger rod 11 engages with the distally directed counter holding surface 13a of the holder 13, and the initial operation of the medicament delivery device is therefore performed. In this example, the delivery member guard 14 is either moved to the retracted position by the cap assembly 2 or just simply being enclosed by the cover of the cap assembly 2.

The inventive concept has mainly been described above with reference to a few examples. However, as is readily appreciated by a person skilled in the art, other embodiments than the ones disclosed above are equally possible within the scope of the inventive concept, as defined by the appended claims.

1-14 (canceled)

15. A method of assembling a medicament delivery device, the method comprising:
providing a housing of the medicament delivery device; wherein the housing (10) extends along a longitudinal axis (L) between a proximal end and a distal end, wherein the distal end of the housing (10) is pointing away from a medicament delivery site during use of the medicament delivery device; and wherein the proximal end of the housing (10) is pointing towards the medicament delivery site during use of the medicament delivery device;
providing a power unit of the medicament delivery device; wherein the power unit comprises a plunger rod (11) and a holder (13) engaged with the plunger rod;
assembling the power unit into the housing (10);
providing a cap assembly (2) extending along the longitudinal axis (L) between a proximal end and a distal end; wherein the cap assembly (2) comprises a cover (20) configured to cover a medicament delivery member of the medicament delivery device;
connecting the cap assembly (2) to either the plunger rod (11) or the holder (13); and
moving the cap assembly (2) relative to the housing (10) in the distal direction of the housing (10) until the cap assembly (2) attaches to the proximal end of the housing (10),
wherein during the step of moving the cap assembly (2) relative to the housing (10), the cap assembly (2) moves:
(i) the plunger rod (11) relative to the housing (10) when the cap assembly (2) is connected to the plunger rod (11); or
(ii) the holder (13) relative to the housing (10) when the cap assembly is connected to the holder (13).

16. The method according to claim 15, wherein before connecting the cap assembly to either the plunger rod or the holder, the method comprises the further steps of:
providing a medicament container; and
inserting the medicament container into the housing from the proximal end of the housing.

17. The method according to claim 15, wherein before assembling the power unit into the housing, the method comprises the further steps of:
providing a delivery member guard; and
assembling both the delivery member guard and the power unit into the housing.

18. The method according to claim 17, wherein the method comprises the further steps of:
engaging the delivery member guard to the holder either before or after assembling both the delivery member guard and the power unit into the housing.

19. The method according to claim 15, wherein the step of moving the plunger rod relative to the housing comprises moving the plunger rod relative to the holder while the holder is immovable relative to the housing.

20. The method according to claim 15, during the step of moving the holder relative to the housing, the plunger rod is immovable relative to the housing.

21. The method according to claim 16, wherein the step of connecting the cap assembly to the plunger rod comprises the steps of:
contacting a proximal end of the medicament container with the cap assembly from the distal end of the cap assembly.

22. The method according to claim 21, wherein the step of moving the plunger rod relative to the holder comprises the steps of:
using the cap assembly to move the medicament container in the distal direction relative to the housing;
contacting a proximal end of the plunger rod with a distal end of the medicament container;
using the distal end of the medicament container to move the plunger rod in the distal direction relative to the housing;
disengaging the plunger rod from the holder by moving the plunger in the distal direction relative to the housing; and
attaching the cap assembly to the proximal end of the housing.

23. The method according to claim 22, wherein the step of connecting the cap assembly to the holder comprises the step of:
moving the cap assembly in the proximal direction relative to the delivery member guard, such that the cap assembly contacts a proximal end of the delivery member guard.

24. The method according to claim 23, wherein the step of moving the plunger rod relative to the holder comprises the steps of:
using the cap assembly to move the delivery member guard in the distal direction relative to the housing;
using the distal movement of the delivery member guard to move the holder relative to the plunger rod; and
attaching the cap assembly to the proximal end of the housing.

25. The method according to claim 24, wherein the step of moving the holder relative to the plunger rod comprises a step of using the distal movement of the delivery member guard to rotate the holder relative to the plunger rod.

26. The method according to claim 24, wherein the step of moving the holder relative to the plunger rod comprises a step of using the distal movement of the delivery member guard to linearly move the holder relative to the plunger rod in a direction transverse to the longitudinal axis.

27. The method according to claim 15, wherein when the cap assembly is attached to the proximal end of the housing, the cap assembly moves axially towards the distal end of the housing relative to the housing until the cap assembly snap-fits to the housing.

28. The method according to claim 16, wherein after the cap assembly is attached to the proximal end of the housing, the medicament delivery device is functionally completed.
